(19) United States
(12) Patent Application Publication
(10) Pub. No.: US 2021/0283415 A1
(43) Pub. Date: Sep. 16, 2021

(54) APPLICATION OF NANOPARTICLES TO TREAT INFECTIONS IN THE RESPIRATORY TRACT

(71) Applicants: Ghassan S Kassab, La Jolla, CA (US); Ali Dabiri, San Diego, CA (US)

(72) Inventors: Ghassan S Kassab, La Jolla, CA (US); Ali Dabiri, San Diego, CA (US)

(21) Appl. No.: 17/202,449

(22) Filed: Mar. 16, 2021

Related U.S. Application Data
(60) Provisional application No. 63/023,215, filed on May 11, 2020.
(60) Provisional application No. 63/008,521, filed on Apr. 10, 2020.
(60) Provisional application No. 62/990,427, filed on Mar. 16, 2020.

(51) Int. Cl. A61N 5/06; A61K 47/69

(52) U.S. Cl. CPC A61N 5/06; A61N 2005/0612; A61N 2005/066; A61K 47/6929

(57) ABSTRACT

A method of using nanoparticle (NP) spray to treat an infection in the respiratory system and far infrared radiation (FIR) to treat inflammation. The method uses a spray probe inserted into the airway of a patient to apply mSiO₂ and FIR to a target site in the upper respiratory tract. FIR applied may be in the 3-10 μm range. The mSiO₂ spray kills any organisms, including COVID-19 and FIR reduces inflammation resulting from infection. The treatment may be applied to intubated or spontaneously breathing patients and via the oral or nasal passages.

### PRIORITY

The present patent application is related to, and claims the priority benefit of, U.S. Provisional Patent Application Ser. No. 62/990,427 filed on Mar. 16, 2020, and is also related to and also claims the priority benefit of, U.S. Provisional Patent Application Ser. No. 63/008,521 filed on Apr. 10, 2020, and is also related to and also claims the priority benefit of, U.S. Provisional Patent Application Ser. No. 63/023,215 filed on May 11, 2020, the contents of which are hereby incorporated by reference in their entirety into this disclosure.

### BACKGROUND

COVID-19 belongs to a family of viruses known as coronaviruses. Named for the crown-like spikes on their surfaces, they infect mostly bats, pigs, and small mammals. This family of viruses mutates easily and can transmit from animal to human, and from human to human. In recent years, coronaviruses have become a growing player in infectious-disease outbreaks worldwide. Seven strains are known to infect humans, including this new virus (COVID-19) which causes illness in the respiratory tract. Some cause common colds while others, by contrast, rank among the deadliest of human infections: Severe acute respiratory syndrome (SARS), and Middle East respiratory syndrome (MERS).

The COVID-19 virus infects the upper and lower respiratory tract and damages the cells that line the respiratory tract at the alveoli level where the exchange of oxygen and carbon dioxide occurs during respiration. As the virus enters the lung cells, it starts to replicate, destroying the cells. Our body senses all viruses as foreign invaders, which triggers the immune system to contain and control the virus and stop it from replicating itself. The immune system response to the COVID-19 can cause inflammation and destroy lung tissue.

Patients initially develop a fever, cough, and aches, and can progress to suffering from shortness of breath and complications from pneumonia. Other reported symptoms include fatigue, sore throat, headache, and nausea, with vomiting and diarrhea. The end result may be pneumonia which means the air sacs or alveoli in the lungs become inflamed and filled with fluid, making it harder to breathe. These symptoms can also make it harder for the lungs to get oxygen to blood, potentially triggering a cascade of respiratory/cardiac complications. The lack of oxygen leads to more inflammation, which causes more problems in the body resulting in the death of liver and kidney cells and eventually the patient dies.

People of all ages have been infected, but the risk of severe disease and death is highest for older people and those with other health conditions such as heart disease, chronic lung disease, cancer, and diabetes. In certain patients with comorbidities, this condition requires urgent medical attention including the use of a ventilator to stabilize the condition of the patient.

Clinically, patients must be placed on ventilators for weeks as they recover from the viral infection. Recent grim data shows that the majority (over 80%) of patients that are placed on ventilators succumb to the disease and die. It is projected the number of patients that will require respirators dwarfs the number of respirators present available in hospitals and ICUs. Hence, there is a substantial need to reduce the duration of use of respirators by speeding up recovery from the infection. To attain this goal, it is critical to treat high risk patients earlier in the disease stage (in the large airways) to prevent progression of the disease to the smaller airways and alveoli.

There is currently no FDA approved treatment for the COVID-19. Drugs approved for malaria such as chloroquine and antibiotics (e.g., Zithromax Z-PAK, azithromycin) are currently in clinical trial for COVID-19. Even if effective, chloroquine is not without the significant side effects that have been seen when the medication is used for malaria which includes blurred vision, nausea, vomiting, abdominal cramps, headache, diarrhea, bleaching of hair and hair loss. Hence, it is important to devise additional therapies that are local and do not have systemic side effects.

Thus, there is a need for effective treatment of COVID-19 that can be applied both before the patient needs to be intubated and during intubation and additionally does not have systemic side effects.

### BRIEF SUMMARY

An objective of the devices and methods referenced herein is to utilize and optimize nanoparticles (NP) for treatment of COVID-19 in the respiratory tract prior to disease progression to the smaller airways in high-risk patients with co-morbidities (e.g., diabetes, hypertension, heart and lung disease, etc.). The NP spray and far infrared light (FIR) fiber optics probes will be introduced through nasal cavity to reach the respiratory tract. The disclosed treatment is novel in the following ways: 1) Safe and effective to reduce viral load, 2) Reduce inflammation in the infrared red light range, 3) No drugs with potential systematic harmful side effects into the body, and 4) Potentially lower treatment cost than drugs. The SiO₂ NP spray/FIR light treatment should not last more than several hours while the patient breathes spontaneously. In another embodiment a nanoparticle probe can be used by itself.

As such, in one embodiment, the present disclosure includes devices, namely a NP probe, configured for integration with a tracheal tube or to traverse an airway independently, that allows transmission of NP such as mesopores SiO₂ (mSiO₂) in the airways that can reach the alveolar sacs where COVID-19 resides. Systems of the present disclosure would therefore include one or more NP probes and one or more other devices or items, such as a tracheal tube, a power source operably coupled to the NP probe to provide power to said NP probe, and the like.

In another embodiment, treatment of the infected patient semi-invasively in a relatively short period can be accomplished by the application of NP to destroy the virus in the respiratory tract and FIR to treat the inflammation directly in the airways. The NP/FIR fiber optics probe(s) will be introduced through the nasal or oral cavity to reach the respiratory tract. The NP spray and FIR can be emitted from a single probe or from two probes.

The NP/FIR light treatment should not last more than several hours and can be applied while the patient breathes spontaneously. The impact of this disclosure is to help combat this pandemic condition that is paralyzing our country. The effect of speeding up treatment to reduce the burden on ventilators and the overall effect on the healthcare system which is substantially burdened cannot be overstated. Additionally, the potential for leading to improved outcomes for the many patients who are currently suffering from COVID-19 is great.

The present disclosure includes disclosure of a NP probe, as described herein.

The present disclosure includes disclosure of a system, comprising a NP probe and another device or item, such as a power source, a tracheal tube, and the like.

The present disclosure includes disclosure of methods of treating a viral infection of the lung using a NP probe to spray NP at or near the location of the virus causing the viral infection in the lung.

The present disclosure includes disclosure of a method, comprising the steps of positioning a NP probe within a trachea or another part of the respiratory system of a mammalian patient so that the portion of the NP probe configured to spray NP is at or near the location of the virus causing the viral infection in the lung, and operating the NP probe to spray NP at or near the location of the virus causing the viral infection in the lung to kill some or all of the virus.

The present disclosure includes disclosure of a method, comprising the steps of inserting a tracheal tube within a trachea or another part of the respiratory system of a mammalian patient, positioning a NP probe within the tracheal tube so that the portion of the NP probe configured to sprayNP at or near the location of the virus causing the viral infection in the lung, and operating the NP probe to spray NP at or near the location of the virus causing the viral infection in the lung to kill some or all of the virus.

The present disclosure includes disclosure of a method, wherein the NP sprayed by the NP probe is NP.

The present disclosure includes disclosure of methods of treating mammalian patients having COVID-19 using an NP spray probe.

The present disclosure also includes apparatuses and methods for treating a respiratory infection using combinations of NP and FIR.

An exemplary method of treating a respiratory infection comprises the steps of: introducing at least one probe into an airway of a patient; advancing the at least one probe to a target site; and activating the at least one probe such that the at least one probe sprays NP.

An exemplary method of treating a respiratory infection comprises the steps of: introducing at least one probe into an airway of a patient; advancing the at least one probe to a target site; activating the at least one probe such that the at least one probe sprays NP and activating the at least one probe such that the at least one probe emits FIR.

An exemplary method of treating a respiratory infection comprises the steps of: positioning a probe within a trachea or another part of the respiratory system of a mammalian patient so that a portion of the probe configured to spray NP is at or near the location of a virus causing the respiratory infection; and operating the probe to spray NP at or near the location of the virus causing the NP to kill some or all of the virus.

An exemplary method of treating a respiratory infection comprises the steps of: positioning a probe within a trachea or another part of the respiratory system of a mammalian patient so that a portion of the probe configured to spray NP and to emit FIR is at or near the location of a virus causing the respiratory infection; and operating the probe to spray NP and emit FIR at or near the location of the virus causing the NP to kill some or all of the virus.

In the exemplary methods for treating a respiratory infection, the FIR emitted is in the 3-10 μm range.

In the exemplary methods for treating a respiratory infection, the NP spray and FIR emission occur simultaneously.

In the exemplary methods for treating a respiratory infection, the at least one probe is introduced into an airway of a patient further through a tracheal tube.

In the exemplary methods for treating a respiratory infection, the NP is sprayed within the airway for a duration of 1-4 hours. In the exemplary methods for treating a respiratory infection, the NP spray and the FIR are emitted for 1-4 hours.

In the exemplary methods for treating a respiratory infection, the target site is the trachea and specifically the upper trachea.

In another embodiment, a method of treating a respiratory infection comprises the step of rotating the portion of the probe configured to spray NP and configured to emit FIR while the probe is operated to spray NP and emit FIR.

An exemplary system for treating a respiratory infection comprises: a NP source; and at least one probe operably connected to the NP source and configured to traverse the airway of an infected patient and configured to spray NP generated by the NP source.

An exemplary system for treating a respiratory infection comprises: a NP source, a FIR source; and at least one probe operably connected to the NP source and the FIR source and configured to traverse the airway of an infected patient and configured to sprayNP and FIR generated by the NP source and the FIR source.

An exemplary system for treating a respiratory infection comprises: a NP source, a FIR source; and a single probe operably connected to the NP source and the FIR source and configured to traverse the airway of an infected patient and configured to emit spray NP and FIR generated by the NP source and the FIR source.

An exemplary method of treating a respiratory infection comprises the steps of: introducing at least one probe into an airway of a patient; advancing the at least one probe to a target site; and activating the at least one probe such that the at least one probe applies nanoparticles to the target site.

An exemplary method of treating a respiratory infection comprises the steps of: introducing at least one probe into an airway of a patient; advancing the at least one probe to a target site; activating the at least one probe such that the at least one probe applies nanoparticles to the target site; and activating the at least one probe such that the at least one probe emits far infrared radiation (FIR).

In an alternate exemplary method of treating a respiratory infection, the FIR emitted is in the 3-10 μm range. In an alternate exemplary method of treating a respiratory infection, the nanoparticles are silica based and can also be mesoporous silica nanoparticles. In an alternate exemplary method of treating a respiratory infection, the nanoparticles are administered at a concentration up to 10⁻⁵ g mL⁻¹.

In an alternate exemplary method of treating a respiratory infection, the step of activating the at least one probe such that the at least one probe emits FIR is performed after the step of activating the at least one probe such that the at least one probe applies nanoparticles to the target site.

In an alternate exemplary method of treating a respiratory infection, the step of applying nanoparticles within the airway lasts for a duration of 1-4 hours.

An exemplary method of treating a respiratory infection comprises the steps of: positioning a probe within a trachea or another part of the respiratory system of a mammalian patient so that a portion of the probe configured to spray nanoparticles is at or near the location of a virus causing the respiratory infection; and operating the probe to apply nanoparticles at or near the location of the virus causing the nanoparticles to destroy some or all of the virus.

An exemplary method of treating a respiratory infection comprises the steps of: positioning a probe within a trachea or another part of the respiratory system of a mammalian patient so that a portion of the probe configured to spray nanoparticles is at or near the location of a virus causing the respiratory infection; and operating the probe to apply nanoparticles at or near the location of the virus causing the nanoparticles to destroy some or all of the virus and a portion of the probe configured to spray nanoparticles is also configured to emit FIR and the method further comprises the step of operating the probe to emit FIR.

In an alternate exemplary method of treating a respiratory infection, the step of operating the probe to emit FIR is performed after the step of operating the probe to apply nanoparticles.

An embodiment of a system for treating a respiratory infection comprises: nanoparticles; and at least one probe configured to traverse the airway of a infected patient and configured to apply nanoparticles to a target site from a distal end of the at least one probe.

An embodiment of a system for treating a respiratory infection comprises: nanoparticles; at least one probe configured to traverse the airway of a infected patient and configured to apply nanoparticles to a target site from a distal end of the at least one probe; and a FIR source and wherein the at least one probe is operably connected to the FIR source and configured to emit FIR generated by the FIR source.

An embodiment of a system for treating a respiratory infection comprises: nanoparticles; a single probe configured to traverse the airway of a infected patient and configured to apply nanoparticles to a target site from a distal end of the single probe; and a FIR source and wherein the single probe is operably connected to the FIR source and configured to emit FIR generated by the FIR source.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a schematic of the interaction of the nanoparticles with the virus and the cell, according to an exemplary embodiment of the present disclosure;

FIG. 2 shows functional and non-functional surfaces, according to an exemplary embodiment of the present disclosure;

FIG. 3 shows a nanoparticle spray probe and a FIR probe inserted through the nasal cavity and progressed to the trachea, according to an exemplary embodiment of the present disclosure; and

FIG. 4 shows embodiments of NP, FIR and a combined NP/FIR probe according to exemplary embodiments of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration.

### DETAILED DESCRIPTION

The present disclosure describes the use of NP spray to treat infections such as by killing viruses in the lung. The present disclosure describes a NP probe which is, in one embodiment, integrated within a tracheal tube to reach the lung directly through intubation when the patient requires a ventilator. Tracheal intubation, usually simply referred to as intubation, is the placement of a flexible plastic tube through the mouth or nostril into the trachea (windpipe) to maintain an open airway or to serve as a conduit through which to administer certain drugs, or as disclosed herein, a device consistent with the present disclosure.

In addition, non-intubated patients may also be treated by the subject of the present disclosure. The NP and/or FIR probe(s) described herein may be introduced nasally or orally and advanced to the treatment site, usually the upper or lower trachea.

The methods and apparatuses disclosed within can treat patients, high-risk or otherwise, in a relatively short period directly at the site of infection to avoid systemic effects. This treatment is novel in that it: 1) reduces viral load directly in the upper airways before it progresses to the smaller bronchi, 2) reduce inflammation in situ in the trachea, 3) provides safe exposure to NP spray, and 4) avoids medications/chemicals with harmful systemic side effects. This therapy can be used beyond COVID-19 and applies to other viruses and influenza strains that claim tens of thousands of lives of patients at high risk every year.

Data indicates that peak COVID-19 RNA concentrations of up to 5×10⁸ copies per swab were reached before day 5 for infected subjects, all being young to middle-aged professionals without significant underlying disease with mild symptoms (1). COVID-19 was later found in sputum (regurgitated from the smaller airways through coughing) at mean concentrations of 1.2-2.8×10⁶ copies per ml (1); i.e., much higher concentrations than the nasal swabs. Although COVID-19 has striking differences from SARS in that successful live virus isolation from throat swabs is possible, COVID-19 resembles SARS in terms of replication in the lower respiratory tract as a result of progression from the upper respiratory tract. This suggests active virus replication in upper conduction respiratory tract tissues that progress distally to the functional (transport) airways (alveolus). This provides the rationale to focus the radiation intervention in the upper respiratory tract in the earlier stage of the disease to mitigate progression to the smaller airways.

A virus is not a living organism but consists of RNA covered by a protective layer of lipid (fat), which, when absorbed by the cells of the ocular, nasal or buccal mucosa, changes their genetic code (mutation) and converts them into an aggressor that multiplies. The immune response to the COVID-19 can also destroy respiratory tract tissues and cause inflammation. Since the virus is not a living organism but a RNA strand, it is not killed; it decays on its own. The disintegration time depends on the temperature, humidity and type of material where it lies. The virus is relatively fragile; the only thing that protects it is a thin outer layer of fat and heat melts fat and disintegrates the virus. NP spray causes nanoparticle binding to viral particles, thus inhibiting viral transduction.

### Nanoparticle Selection

Scientists have been using nanoparticles to control viruses for decades (2). One of the important design requirements is to select an effective nanoparticle that is safe for duration of exposure time (on the scale of hours) without any harmful effects. These types of nanoparticles can feature a well-defined and tunable porosity at the nanometer scale, high loading capacity, and multiple functionality for targeting and entering different types of cells. Nanosized mesoporous silica particles with high colloidal stability attract growing attention as drug delivery systems for targeted cancer treatment and as bioimaging devices (3). Silver nanoparticles have demonstrated efficient inhibitory activities against human immunodeficiency virus (HIV) (4) and hepatitis B virus (HBV) (5). The inhibitory effects of silver nanoparticles on influenza A virus may be a novel clinical strategy for the prevention of influenza virus infection during the early dissemination stage of the virus (6). Gold nanoparticles have shown high activity towards the inhibition of influenza virus infection (7). Porous Si nanoparticles could also be used for efficient delivery of antivirals to infected cell from influenza A virus (8).

Silva et al (2) designed experiments to address the mechanism of antiviral action of the nanoparticles and found that their hydrophobic/hydrophilic characters play a crucial role. The results revealed that the use of functionalized silica particles is a promising approach for controlling viral infection and offered promising strategies for viral control. The mesoporous SiO₂ (mSiO₂) particles interactions with a specific virus envelope are the basis of the antiviral activity of the mSiO₂ particles, as stronger virus-mSiO₂ bonds disturb the attachment of cell receptors to the virus envelope, and the viral transduction ability is reduced. They showed that nanoparticles exhibited no significant toxicity to mammalian cells, while observing up to 50% decline in the viral transduction of two distinct recombinant viruses. The results demonstrated for the first time the antiviral activity of mSiO₂ particles and allow them to propose a mechanism of mSiO₂ particles antiviral action based on surface interactions between silica, cells and viruses. Thus, this study provides new insights to the development of innovative strategies for viral control and for anti-HIV therapy that could also explore the possibility of loading antiviral drugs into the mSiO₂ particles mesopores.

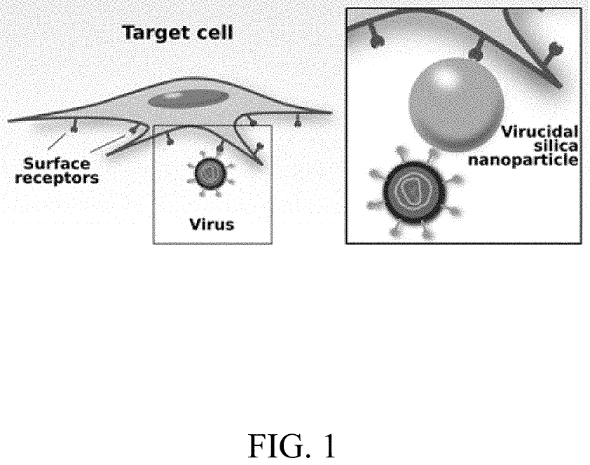

Tailored nanostructures have emerged as a promising alternative for virus control (9,10) since they have shown activity against different viruses, including hepatitis B (4,5)virus, human immunodeficiency virus(11,12), herpes simplex virus(13), respiratory syncytial virus(13), adenovirus (14), monkey pox virus (13), influenza (7), and H1N1 influenza A viruses (6,15,16). However, not much is known about the nanostructures mechanisms of antiviral action (FIG. 1).

mSiO₂ was selected as the candidate nanoparticle for the following reasons: 1) The nanoparticles allow conjugation with biomolecules like proteins and DNA (17), 2) They have proved to be highly useful for biosensing, assay labelling, bioimaging, and in research on a variety of molecular tags in cellular and molecular biology(17,18).

Preparation and Characterization of mSiO₂ Nanoparticles

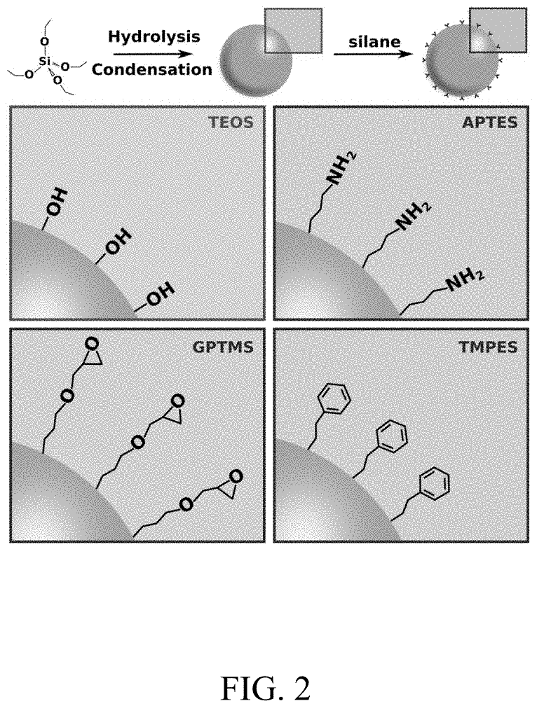

The interaction of nanoparticles with cells and viruses depends on the surface properties of the nanoparticles and the body under study. Silva et al. (1) prepared nanoparticles by the hydrolysis and condensation of tetraethylorthosilicate (TEOS) under basic conditions (top of FIG. 2). After the hydrolysis and condensation of TEOS, silica particles exhibited silanol groups Si—OH on their surfaces (top left box in FIG. 2). Organic groups were grafted onto the surface of the silica nanoparticles by adding organofunctional alkoxysilane to the reaction medium. After the addition of (3-aminopropyl) triethoxysilane (APTES), (3-glycidyloxypropyl) trimethoxysilane (GPTMS), and trimethoxy(2-phenylethyl)silane (TMPES), four silica surfaces were prepared respectively, aminopropyl, glycidyloxypropyl, and phenylethyl groups, and OH groups (FIG. 2). They were named SiO₂-TEOS, mSiO₂-APTES, mSiO₂-GPTMS, and mSiO₂-TMPES.

The synthesized particles had a radius of a few hundred nanometers and exhibited uniform spherical shape and smooth surfaces. They were characterized including morphology, particle size distribution and cytotoxicity and inhibitory potential to prevent viral transduction. All four mSiO₂ nanoparticles were uniform in shape and had similar sizes with small differences in size. The most noticeable difference between all particles was related to the particle surface properties. The degree of functionalization contributed to the zeta-potential differences between particles (zeta-potential is the potential difference between the dispersion medium and the stationary layer of fluid attached to the dispersed particle). In addition, the mesopores diameter and the silanolates on their surfaces may contribute to the final zeta-potential. No cytotoxic effect was observed to the cells tested when particles concentration was equal to or under 10⁻⁵ g mL⁻¹ and were still able to reduce transduction of a recombinant virus harboring VSV-G (Vesicular Stomatitis Virus Glycoprotei envelope).

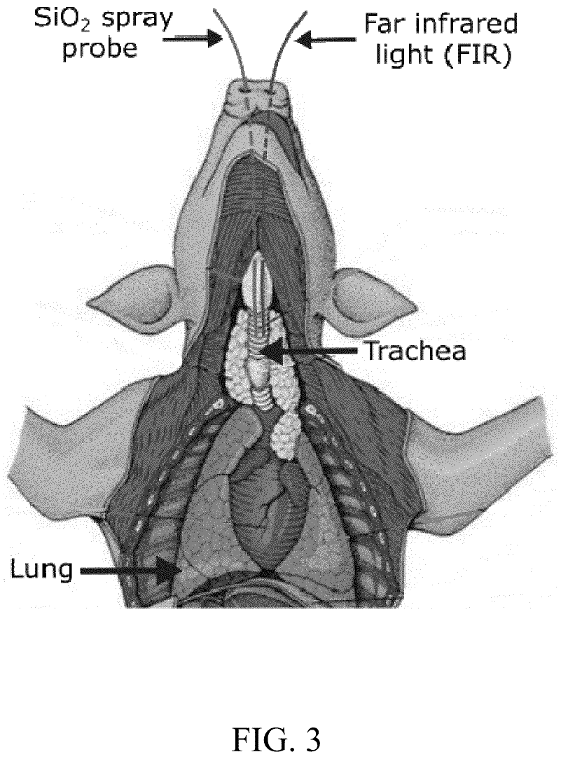

The functionality of all four groups is the same in terms of reducing the viral load. Nanoparticles must be prepared in the form of a spray gun probe that can be inserted through the nasal cavity as depicted in FIG. 3.

### Treatment Methods

It is a goal of this disclosure to control COVID-19 utilizing the methods and devices described herein such that a resulting reduction in viral load along with a reduction in inflammation will lead to speeder recovery. Hence, the objective is to apply nanoparticles for a short duration directly in the airways to reduce the viral load followed by FIR to reduce inflammation.

The present disclosure describes the application of nanoparticles followed by FIR directly into the airways earlier in the infection process to reduce the viral load and combat inflammation to mitigate the progression of the etiology of the infection to lower respiratory tract that impacts gas exchange. Nanoparticles can be applied for a short exposure time to kill the virus and FIR in the wavelength range of 3-10 μm can be applied to reduce inflammation. The exposure time will depend on concentration of the nanoparticles. The proposed nanoparticle exposure is safe and avoids damage to tissue.

Infrared therapy is an effective and safe remedy for pain and inflammation (19). Since infrared therapy enhances and improves circulation in the skin and other parts of the body, it can bring oxygen and nutrients to injured tissues, promoting healing. Other studies indicate that FIR therapy is effective in relieving pain in patients with chronic pain, chronic fatigue syndrome, and fibromyalgia (20).

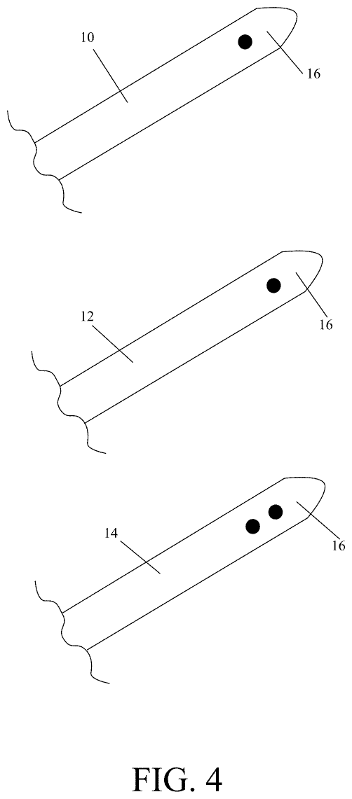

As depicted in FIGS. 3-4, an FIR fiber optics probe 10 is inserted through the nasal cavity and progresses to the trachea as depicted in FIG. 3. The small diameter probe can be advanced to the region of interest through the use of fluoroscopy. An adult's trachea has an inner diameter of about 1.5 to 2 cm (0.59 to 0.79 in) and a length of about 10 to 11 cm (3.9 to 4.3 in); wider in males than females. It begins at the bottom of the larynx, and ends at the carina, the point where the trachea branches into left and right main bronchi. The inner diameter is smaller for children. The diameter of the fiber optic probe is significantly smaller (3 mm or less) and hence will not interfere with respiration in the relatively larger diameter trachea. In summary, the FIR fiber optics technology has the following advantages. 1) Significantly smaller diameter than trachea, 2) Flexible, and 3) The electrical connections to power the probes are placed outside of the body. A NP probe 12 of similar configuration (size, flexibility, etc) is also inserted into the airway. The NP probe 12 should be a form of a spray gun probe. Both probes are advanced to a target site where the NP spray and FIR emission will occur. In an alternate embodiment, the NP probe and FIR probe can be combined into a single probe 14 configured to perform both functions, emit FIR and spray NP.

In this embodiment, the two probes are configured to spray NP and emit FIR from their distal end 16. Once the distal end 16 of the probes are advanced to the target site, the probes are activated to spray the NP and emit FIR. The spray of NP lasts a short duration, between 1-4 hours, followed by emission of FIR which can last 1-4 hours. During the duration of treatment, the nanoparticles concentration can start from 50% of the maximum concentration (10^{-s} g mL^{-g}) and increase to 100%.

It is within the scope of this disclosure that the insertion of probes can be achieved nasally or orally and can be performed on either an intubated or non intubated patient.

While various embodiments of devices, systems, and methods to kill coronavirus in the lung using nanoparticles and far infrared radiation have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

For example, although the specification refers to treating COVID-19, it is within the scope of the disclosure that other types of coronavirus or, in general, other viruses and diseases susceptible to nanoparticles could be treated by the methods and apparatus disclosed. It is also envisioned that other locations in the body could be treated. For instance, the trachea is specifically referred to, but other portions of the lungs or other luminal organs could be the target for treatment.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and remain within the scope of the present disclosure.

### REFERENCES

1. R. Wölfel, et al., Virological assessment of hospitalized patients with COVID-2019, Nature, Apr. 1, 2020. Nature https://doi.org/10.1038/s41586-020-2196, 2020.
2. Juliana Martins de Souza e Silva, Talita Diniz Melo Hanchuk, Murilo Izidoro Santos, Jörg Kobarg, Marcio Chaim Bajgelman, and Mateus Borba Cardoso, Viral Inhibition Mechanism Mediated by Surface-Modified Silica Nanoparticles, ACS Appl. Mater. Interfaces, 2016, 8, 16564-16572.
3. Christian Argyo, Veronika Weiss, Christoph Bräuchle, Thomas Bein, Multifunctional Mesoporous Silica Nanoparticles as a Universal Platform for Drug Delivery, Chem. Mater. 2014, 26, 1, 435-451 https://doi.org/10.1021/cm40259t.
4. Carja, G.; Grosu, E. F.; Petrarean, C.; Nichita, N. Self-assemblies of plasmonic gold/layered double hydroxides with highly efficient antiviral effect against the hepatitis B virus. Nano Res. 2015, 8 (11), 3512-3523.
5. Lu, L.; Sun, R. W. Y.; Chen, R.; Hui, C. K.; Ho, C. M.; Luk, J. M.; Lau, G. K. K.; Che, C. M. Silver nanoparticles inhibit hepatitis B virus replication. Antivir. Ther. 2008, 13 (2), 253-262.
6. Xiang, D. X.; Chen, Q.; Pang, L.; Zheng, C. L. Inhibitory effects of silver nanoparticles on H1N1 influenza A virus in vitro. J. Virol. Methods 2011, 178 (1-2), 137-142.
7. Papp, I.; Sieben, C.; Ludwig, K.; Roskamp, M.; Bottcher, C.; Schlecht, S.; Herrmann, A.; Haag, R. Inhibition of Influenza Virus Infection by Multivalent Sialic-Acid-Functionalized Gold Nanoparticles. Small 2010, 6 (24), 2900-2906.
8. L. Bimbo et al., Inhibition of Influenza A Virus Infection in Vitro by Saliphenylhalamide-Loaded Porous Silicon Nanoparticles, ACS Nano 2013, 7, 8, 6884-6893.
9. Lembo, D.; Cavalli, R. Nanoparticulate delivery systems for antiviral drugs. Antivir Chem. Chemother 2010, 21 (2), 53-70.
10. Rai, M.; Deshmukh, S. D.; Ingle, A. P.; Gupta, I. R.; Galdiero, M.; Galdiero, S. Metal nanoparticles: The protective nanoshield against virus infection. Crit. Rev. Microbiol. 2016, 42 (1), 46-56.
11. Lara, H. H.; Ixtepan-Turrent, L.; Garza Trevino, E. N.; Singh, D. K. Use of silver nanoparticles increased inhibition of cell-associated HIV-1 infection by neutralizing antibodies developed against HIV-1 envelope proteins. J. Nanobiotechnol. 2011, 9, 38.
12. Senanayake, T. H.; Gorantla, S.; Makarov, E.; Lu, Y.; Warren, G.; Vinogradov, S. V. Nanogel-Conjugated Reverse Transcriptase Inhibitors and Their Combinations as Novel Antiviral Agents with Increased Efficacy against HIV-1 Infection. Mol. Pharmaceutics 2015, 12 (12), 4226-4236.
13. Galdiero, S.; Falanga, A.; Vitiello, M.; Cantisani, M.; Marra, V.; Galdiero, M. Silver Nanoparticles as Potential Antiviral Agents. Molecules 2011, 16 (10), 8894-8918.
14. Park, S.; Park, H. H.; Kim, S. Y.; Kim, S. J.; Woo, K.; Ko, G. Antiviral Properties of Silver Nanoparticles on a Magnetic Hybrid Colloid. Appl. Environ. Microb 2014, 80 (8), 2343-2350.
15. Botequim, D.; Maia, J.; Lino, M. M. F.; Lopes, L. M. F.; Simoes, P. N.; Ilharco, L. M.; Ferreira, L. Nanoparticles and Surfaces Presenting Antifungal, Antibacterial and Antiviral Properties. Langmuir 2012, 28 (20), 7646-7656.
16. Levina, A. S.; Repkova, M. N.; Mazurkova, N. A.; Zarytova, V. F. Nanoparticle-Mediated Nonviral DNA Delivery for Effective Inhibition of Influenza a Viruses in Cells. IEEE Trans. Nanotechnol. 2016, 15 (2), 248-254. https://findme10.com/best-coronavirus-uv-light/.
17. D. Knopp et al., Review: Bioanalytical applications of biomolecule-functionalized nanometer-sized doped silica particles, Analytica Chimica Acta, 647, 2009, Pages 14-30.
18. Y. Piao et al., Designed Fabrication of Silica-Based Nanostructured Particle Systems for Nanomedicine Applications, Adv. Func. Materials, 2008, https://doi.org/10.1002/adfm.200800731.
19. Shanshan Shui, Xia Wang, John Y Chiang, and Lei Zheng, Far-infrared therapy for cardiovascular, autoimmune, and other chronic health problems: A systematic review, Exp Biol Med (Maywood), 240(10): 1257-1265, 2015.
20. Everett M. Lautin, and Suzanne M. Levine, Use of Near-Infrared Light Source to Treat Pain, Practical Pain Management, 2012.

1. A method of treating a respiratory infection comprising the steps of:
introducing at least one probe into an airway of a patient;
advancing the at least one probe to a target site; and
activating the at least one probe such that the at least one probe applies nanoparticles to the target site.

2. A method of treating a respiratory infection as in claim 1, further comprising the steps of activating the at least one probe such that the at least one probe emits far infrared radiation (FIR).

3. A method of treating a respiratory infection as in claim 2, wherein the FIR emitted is in the 3-10 μm range.

4. A method of treating a respiratory infection as in claim 1, wherein the nanoparticles are silica based.

5. A method of treating a respiratory infection as in claim 4, wherein the nanoparticles are mesoporous silica nanoparticles.

6. A method of treating a respiratory infection as in claim 2 wherein the step of activating the at least one probe such that the at least one probe emits FIR is performed after the step of activating the at least one probe such that the at least one probe applies nanoparticles to the target site.

7. A method of treating a respiratory infection as in claim 1, wherein the nanoparticles are administered at a concentration up to 10⁻⁵ g mL⁻¹.

8. A method of treating a respiratory infection as in claim 1, further comprising the step of applying nanoparticles within the airway for a duration of 1-4 hours.

9. A method of treating a respiratory infection as in claim 1, wherein the step of activating the at least one probe such that the at least one probe applies nanoparticles to the target, destroys COVID-19 viral particles at the target site.

10. A method of treating a respiratory infection, comprising the steps of:
positioning a probe within a trachea or another part of the respiratory system of a mammalian patient so that a portion of the probe configured to spray nanoparticles is at or near the location of a virus causing the respiratory infection;
operating the probe to apply nanoparticles at or near the location of the virus causing the nanoparticles to destroy some or all of the virus.

11. A method of treating a respiratory infection as in claim 10, wherein the portion of the probe configured to spray nanoparticles is also configured to emit FIR and the method further comprises the step of operating the probe to emit FIR.

12. A method of treating a respiratory infection as in claim 11, wherein the step of operating the probe to emit FIR is performed after the step of operating the probe to apply nanoparticles.

13. A method of treating a respiratory infection as in claim 11, wherein the nanoparticles are administered at a concentration of up to 10⁻⁵ g mL⁻¹.

14. A method of treating a respiratory infection as in claim 13, wherein the nanoparticles are applied for a duration of 1-4 hours.

15. A method of treating a respiratory infection as in claim 13, wherein the nanoparticles are silica based.

16. A method of treating a respiratory infection as in claim 15, wherein the nanoparticles are mesoporous silica nanoparticles.

17. A method of treating a respiratory infection as in claim 11, further comprising the step of rotating the portion of the probe configured to apply nanoparticles and configured to emit FIR while the probe is operated to apply nanoparticles and FIR.

18. A system for treating a respiratory infection comprising:
nanoparticles; and
at least one probe configured to traverse the airway of a infected patient and configured to apply nanoparticles to a target site from a distal end of the at least one probe.

19. A system for treating a respiratory infection as in claim 18, further comprising a FIR source and wherein the at least one probe is operably connected to the FIR source and configured to emit FIR generated by the FIR source.

20. A system for treating a respiratory infection as in claim 18, wherein the at least one probe comprises a single probe configured to emit FIR and apply nanoparticles.
